(19) United States
(12) Patent Application Publication
(10) Pub. No.: US 2025/0223488 A1
(43) Pub. Date: Jul. 10, 2025

(54) DEGRADABLE LOST CIRCULATION MATERIAL FOR PROTECTION OIL AND GAS RESERVOIRS, AND PREPARATION METHOD AND USE THEREOF

(71) Applicant: CHINA UNIVERSITY OF PETROLEUM (EAST CHINA), Qingdao City (CN)

(72) Inventors: Xin ZHAO, Qingdao City (CN); Wenzhi SU, Qingdao City (CN); Zhengsong QIU, Qingdao City (CN); Zhengkai LIU, Qingdao City (CN); Zhiyuan WANG, Qingdao City (CN); Kai SHAN, Qingdao City (CN); Hanyi ZHONG, Qingdao City (CN)

(21) Appl. No.: 18/824,241

(22) Filed: Sep. 4, 2024

(30) Foreign Application Priority Data
Jan. 10, 2024 (CN) 202410032338.9

(51) Int. Cl. C09K 8/508; C07D 405/14; C08G 59/32; C08G 59/42; C08G 59/50; C08G 59/58; C09K 8/035; E21B 21/00

(52) U.S. Cl. CPC C09K 8/5086; C07D 405/14; C08G 59/3245; C08G 59/4238; C08G 59/5073; C08G 59/58; C09K 8/035; E21B 21/003

(57) ABSTRACT

Disclosed are a degradable lost circulation material for protecting oil and gas reservoirs, and a preparation method and use thereof, belonging to the technical field of petrochemicals. The degradable lost circulation material includes an epoxidized vanillin Schiff base compound and a composite curing agent, the composite curing agent including an anhydride curing agent and an imidazole curing agent, wherein the epoxidized vanillin Schiff base compound has a structure shown in formula I.

### CROSS REFERENCE TO RELATED APPLICATION

This patent application claims the benefit and priority of Chinese Patent Application No. 202410032338.9 filed with the China National Intellectual Property Administration on Jan. 10, 2024, entitled “Degradable lost circulation material for protecting oil and gas reservoirs, and preparation method and use thereof”, the disclosure of which is incorporated by reference herein in its entirety as part of the present application.

### TECHNICAL FIELD

The present disclosure belongs to the technical field of petrochemicals, and specifically relates to a degradable lost circulation material for protecting oil and gas reservoirs, and a preparation method and use thereof.

### BACKGROUND

The lost circulation refers to the leakage of a working fluid in a wellbore such as a drilling fluid or a well cementing slurry into stratum under the action of a pressure difference during a downhole operation process such as well drilling, well cementing, and testing. The lost circulation not only causes a heavy economic loss and prolongs an operation cycle, but also leads to complicated situations such as well kicks, well blowouts, and well collapses, and may even cause the scrapping of oil and gas wells. However, due to the presence of fractures as natural loss channels in fractured stratums, an operation process in these fractured stratums is at a high loss risk and has characteristics such as a high loss speed and a low single-plugging efficiency, which is currently one of the key technical problems restricting the safe and efficient drilling operation. [0004] Fractured reservoirs are widely distributed. During a drilling operation for such reservoirs, there is a high risk of lost circulation due to the development of natural fractures, and the lost circulation would cause solid particles and liquid phases in drilling fluids to enter reservoirs in a large quantity and block oil and gas seepage channels such as fractures and bore throats, resulting in serious reservoir damages. Even if the remediation is conducted subsequently through production increase transformations such as flowback and acidification, the blockage by solid particles deep in pores and fractures cannot be completely addressed, and it is difficult to effectively restore the permeability of a reservoir. In order to avoid or reduce an economic loss and a reservoir damage caused by lost circulation, a large quantity of a lost circulation material needs to be added to a drilling fluid to form an effective plugging layer to plug loss channels. Commonly-used lost circulation materials include rigid particles, elastic particles, fiber materials, or the like. After a drilling operation for a reservoir section is completed, it is necessary to allow the self-degradation of a lost circulation material in-situ or the destruction of a plugging layer formed during a plugging process through measures such as flowback and acidification to remove the plugging and restore the oil and gas seepage channels, which requires the lost circulation material to have self-degradation or plugging-removal performance. Some scholars have conducted studies on self-degradable lost circulation materials, including polylactic acid, polymer resin materials, or the like. However, these materials degrade rapidly at high temperature with a degradation rate difficult to control, and exhibit temperature resistance generally at 130° C. or lower, which could not meet use requirements of high-temperature reservoirs. Currently, the degradable lost circulation materials that allow the plugging removal with an acid solution are mainly calcium carbonate, acid-soluble alloys, or the like, which have problems such as a too-high density and insufficient plugging adaptability, and therefore could not fully meet the actual needs of plugging for fractured reservoirs.

### SUMMARY

In view of this, an object of the present disclosure is to provide a degradable lost circulation material for protecting oil and gas reservoirs, and a preparation method and use thereof. The degradable lost circulation material according to the present disclosure has a low density, and exhibits excellent degradability, compressive strength, suspension stability, and plugging and pressure-bearing effects, and great self-adaptability, and could meet the use requirements of plugging and its removal for high-temperature fractured reservoirs.

To achieve the above object, the present disclosure provides the following technical solutions:
The present disclosure provides a degradable lost circulation material, including an epoxidized vanillin Schiff base compound and a composite curing agent,
where the epoxidized vanillin Schiff base compound has a structure shown in formula I:

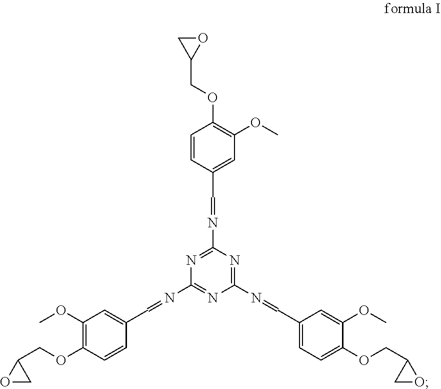 [SMILES: COc1cc(C=Nc2nc(N=Cc3ccc(OCC4CO4)c(OC)c3)nc(N=Cc3ccc(OCC4CO4)c(OC)c3)n2)ccc1OCC1CO1]

and
the composite curing agent includes an anhydride curing agent and an imidazole curing agent.

In some embodiments, a molar ratio of the epoxidized vanillin Schiff base compound to the composite curing agent is in a range of (40-50):(3-28).

In some embodiments, the anhydride curing agent includes at least one selected from the group consisting of phthalic anhydride, trimellitic anhydride, hexahydro-4-methylphthalic anhydride, methylhexahydrophthalic anhydride, and methyltetrahydrophthalic anhydride.

In some embodiments, the imidazole curing agent includes at least one selected from the group consisting of 1-methylimidazole, 2-ethyl-4-methylimidazole, 2-undecylimidazole, and 2-phenylimidazole.

In some embodiments, a molar ratio of the anhydride curing agent to the imidazole curing agent is in a range of (1-5):(1-4).

In some embodiments, the epoxidized vanillin Schiff base compound is prepared by a process including the following steps:
subjecting vanillin and tripolycyanamide to aldehyde-amine condensation reaction in a solution, to obtain a vanillin Schiff base intermediate,
where the vanillin Schiff base intermediate has a structure shown in formula II:

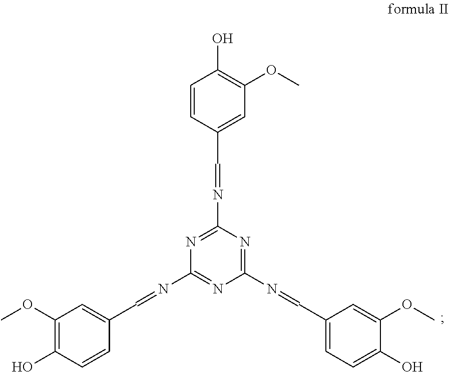

mixing the vanillin Schiff base intermediate, epichlorohydrin, and a first catalyst, and subjecting a resulting mixture to a first epoxidation reaction, to obtain a first epoxidation product; and
mixing the first epoxidation product with a second catalyst, and subjecting a resulting mixture to a second epoxidation reaction, to obtain the epoxidized vanillin Schiff base compound.

In some embodiments, the aldehyde-amine condensation reaction is conducted at a temperature of 70° C. to 90° C. for 24 h to 96 h.

In some embodiments, the first epoxidation reaction is conducted at a temperature of 70° C. to 95° C. for 6 h to 9 h.

The present disclosure also provides a method for preparing the degradable lost circulation material as described in the above technical solutions, including the following steps: [0024] mixing the epoxidized vanillin Schiff base compound and the composite curing agent, and subjecting a resulting mixture to curing reaction, to obtain a glass-like polymer; and
crushing and granulating the glass-like polymer, to obtain the degradable lost circulation material.

The present disclosure also provides use of the degradable lost circulation material as described in the above technical solutions or the degradable lost circulation material prepared by the method as described in the above technical solutions in plugging for a high-temperature fractured reservoir.

The present disclosure provides a degradable lost circulation material, including an epoxidized vanillin Schiff base compound and a composite curing agent, wherein the composite curing agent includes an anhydride curing agent and an imidazole curing agent. The degradable lost circulation material according to the present disclosure has a low density, and thus could avoid the sedimentation problem of a high-density lost circulation material, and exhibits excellent suspension stability in a drilling fluid. Also, the degradable lost circulation material exhibits a high strength, high-temperature resistance, and great pressure-bearing capacity. A Schiff base bond in the epoxidized vanillin Schiff base compound could be hydrolyzed under acid/alkali conditions, and thus the degradable lost circulation material could be degraded under acidic and alkaline conditions, which could meet the use requirements of plugging and its removal for high-temperature fractured reservoirs.

### DETAILED DESCRIPTION OF THE EMBODIMENTS

The present disclosure provides a degradable lost circulation material, including an epoxidized vanillin Schiff base compound and a composite curing agent, wherein
the epoxidized vanillin Schiff base compound has a structure shown in formula I:

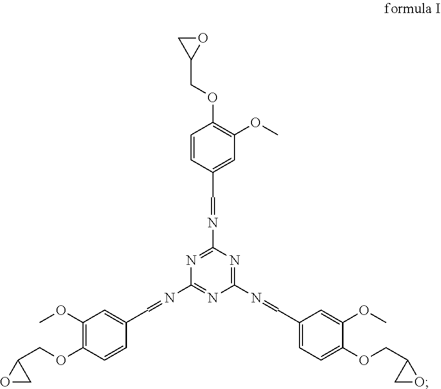 [SMILES: COc1cc(C=Nc2nc(N=Cc3ccc(OCC4CO4)c(OC)c3)nc(N=Cc3ccc(OCC4CO4)c(OC)c3)n2)ccc1OCC1CO1]

and
the composite curing agent includes an anhydride curing agent and an imidazole curing agent.

In the present disclosure, unless otherwise specified, there are no special requirements for sources of raw materials used, and commercially-available products well known to those skilled in the art may be adopted.

The degradable lost circulation material according to the present disclosure includes a composite curing agent. In the present disclosure, the composite curing agent includes an anhydride curing agent and an imidazole curing agent. In some embodiments, a molar ratio of the anhydride curing agent to the imidazole curing agent is in a range of (1-5):(1-4) and preferably 4:3.

In some embodiments of the present disclosure, the anhydride curing agent includes at least one selected from the group consisting of phthalic anhydride, trimellitic anhydride, hexahydro-4-methylphthalic anhydride, methylhexahydrophthalic anhydride, and methyltetrahydrophthalic anhydride, and is preferably trimellitic anhydride and/or methylhexahydrophthalic anhydride, or hexahydro-4-methylphthalic anhydride. In some embodiments of the present disclosure, when the anhydride curing agent is a combination of two or more of the above, there is no special restriction on a ratio among the different anhydride curing agents, and any ratio may be adopted.

In some embodiments of the present disclosure, the imidazole curing agent includes at least one selected from the group consisting of 1-methylimidazole, 2-ethyl-4-methylimidazole, 2-undecylimidazole, and 2-phenylimidazole, and is preferably 1-methylimidazole and/or 2-ethyl-4-methylimidazole, 1-methylimidazole and/or 2-phenylimidazole. In some embodiments of the present disclosure, when the imidazole curing agent is a combination of two or more of the above, there is no special restriction on a ratio among different imidazole curing agents, and any ratio may be adopted.

The degradable lost circulation material according to the present disclosure includes an epoxidized vanillin Schiff base compound.

In some embodiments of the present disclosure, a molar ratio of the epoxidized vanillin Schiff base compound to the composite curing agent is in a range of (40-50):(3-28) and preferably (40-45):(5-25).

The anhydride curing agent is an epoxy resin curing agent. A cured material produced with the anhydride curing agent has a high strength, a low high-temperature shrinkage rate, and excellent mechanical properties, but exhibits poor medium resistance (especially alkali resistance) and poor heat and humidity resistance, and thus the cured material could be degraded under acid/alkali conditions. The imidazole curing agent could undergo addition reaction with an epoxy resin by virtue of active hydrogen in secondary amino, and could also serve as an anionic polymeric curing agent to cure an epoxy resin by virtue of nitrogen atom in tertiary amino. Therefore, it could promote and strengthen a curing effect of the anhydride curing agent and further enhance the temperature resistance and other properties of the degradable lost circulation material.

In some embodiments of the present disclosure, the epoxidized vanillin Schiff base compound is prepared by a process including the following steps:
subjecting vanillin and tripolycyanamide to aldehyde-amine condensation reaction in a solution, to obtain a vanillin Schiff base intermediate; wherein
the vanillin Schiff base intermediate has a structure shown in formula II:

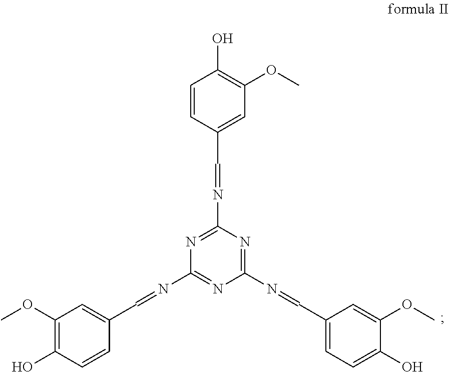

mixing the vanillin Schiff base intermediate, epichlorohydrin, and a first catalyst, and subjecting a resulting mixture to a first epoxidation reaction, to obtain a first epoxidation product; and
mixing the first epoxidation product with a second catalyst, and subjecting a resulting mixture to a second epoxidation reaction, to obtain the epoxidized vanillin Schiff base compound.

In some embodiments of the present disclosure, vanillin and tripolycyanamide are subjected to aldehyde-amine condensation reaction in a solution to obtain a vanillin Schiff base intermediate.

In some embodiments of the present disclosure, a molar ratio of the vanillin to the tripolycyanamide is in a range of (2-5):1 and preferably (3-3.5):1.

In some embodiments of the present disclosure, subjecting vanillin and tripolycyanamide to aldehyde-amine condensation reaction in the solution is performed as follows: mixing a vanillin solution with a tripolycyanamide solution, and subjecting a resulting mixture to the aldehyde-amine condensation reaction. In some embodiments, a solvent used in the vanillin solution is ethanol, glacial acetic acid, or deionized water at 90° C. and preferably deionized water at 90° C. In some embodiments, a solvent used in the tripolycyanamide solution is ethanol, ethylene glycol, or deionized water at 90° C. and preferably deionized water at 90° C. In some embodiments, a molarity of vanillin in the vanillin solution is in a range of 1 mol/L to 3 mol/L and preferably 2 mol/L. In some embodiments, a molarity of tripolycyanamide in the tripolycyanamide solution is in a range of 0.2 mol/L to 0.5 mol/L and preferably 0.3 mol/L to 0.4 mol/L.

In some embodiments of the present disclosure, the aldehyde-amine condensation reaction is conducted at a temperature of 70° C. to 90° C. and preferably 80° C. to 90° C. In some embodiments, the aldehyde-amine condensation reaction is conducted for 24 h to 96 h and preferably 24 h to 80 h.

In some embodiments of the present disclosure, after the aldehyde-amine condensation reaction is completed, a resulting system produced from the aldehyde-amine condensation reaction is subjected to solid-liquid separation, water-washing, and drying sequentially to obtain the vanillin Schiff base intermediate. In some embodiments of the present disclosure, the solid-liquid separation is performed by filtration. In some embodiments, the water-washing is conducted at a temperature of 85° C. to 100° C. and preferably 90° C. In some embodiments, the water-washing is conducted 3 to 5 times and preferably 4 times. In some embodiments, the water-washing is conducted with deionized water. In some embodiments, the drying is performed by vacuum-drying. In some embodiments, the drying is conducted at a temperature of 70° C. to 90° C. and preferably 80° C. In some embodiments, the drying is conducted for 16 h to 32 h and preferably 20 h to 24 h. In some embodiments, the vacuum-drying is conducted at a vacuum degree of 0.05 MPa to 0.1 MPa and preferably 0.06 MPa to 0.09 MPa.

In some embodiments of the present disclosure, the water-washing is conducted to remove the unreacted vanillin and unreacted tripolycyanamide in the system produced from the aldehyde-amine condensation reaction.

In some embodiments of the present disclosure, after the vanillin Schiff base intermediate is obtained, the vanillin Schiff base intermediate, epichlorohydrin, and a first catalyst are mixed, and a resulting mixture is subjected to a first epoxidation reaction, to obtain a first epoxidation product.

In some embodiments of the present disclosure, the first catalyst includes at least one selected from the group consisting of tetrabutylammonium bromide, tetramethylammonium bromide, and succinic anhydride, and is preferably tetramethylammonium bromide. In some embodiments of the present disclosure, when the first catalyst is a combination of two or more of the above, there is no special restriction on a ratio among the different catalysts, and any ratio may be adopted.

In some embodiments of the present disclosure, a molar ratio of the vanillin Schiff base intermediate, the epichlorohydrin, and the first catalyst is in a range of (8-20):(5-15):(1-7) and preferably 17:9:3.

In some embodiments of the present disclosure, the first epoxidation reaction is conducted at a temperature of 70° C. to 95° C. and preferably 88° C. In some embodiments, the first epoxidation reaction is conducted for 6 h to 9 h and preferably 6.5 h to 8 h.

In some embodiments of the present disclosure, after the first epoxidation product is obtained, the first epoxidation product is mixed with a second catalyst, and a resulting mixture is subjected to a second epoxidation reaction, to obtain the epoxidized vanillin Schiff base compound.

In some embodiments of the present disclosure, the second catalyst is an alkali liquor. In some embodiments, a volume ratio of the first epoxidation product to the alkali liquor is is in a range of (1-2):1 and preferably 1:1. In some embodiments, the alkali liquor is a sodium hydroxide solution. In some embodiments, a mass concentration of the sodium hydroxide solution is in a range of 15% to 25% and preferably 20% to 23%. In some embodiments, the second epoxidation reaction is conducted at a temperature of 50° C. to 75° C. and preferably 60° C. to 70° C. In some embodiments, the second epoxidation reaction is conducted for 4 h to 8 h and preferably 5 h to 7 h.

In some embodiments of the present disclosure, after the second epoxidation reaction is completed, a system produced from the second epoxidation reaction is subjected to solid-liquid separation, water-washing, and rotary evaporation to obtain the epoxidized vanillin Schiff base compound.

In some embodiments of the present disclosure, the solid-liquid separation is performed by filtration. In some embodiments, the water-washing is conducted with deionized water. In some embodiments, the water-washing is conducted 3 to 5 times and preferably 4 times. In some embodiments, the rotary evaporation is conducted at a temperature of 20° C. to 60° C. and preferably 40° C. to 50° C. In some embodiments, the rotary evaporation is conducted for 1 h to 3 h and preferably 2 h. In some embodiments, the rotary evaporation is conducted at a vacuum degree of 0.05 MPa to 0.1 MPa and preferably 0.06 MPa to 0.09 MPa. In some embodiments, a product obtained after the water-washing is a viscous liquid that is insoluble in water, and the viscous liquid is subjected to rotary evaporation to remove water, to obtain a final product. [0059] In some embodiments of the present disclosure, the epoxidized vanillin Schiff base compound is synthesized according to the following route:

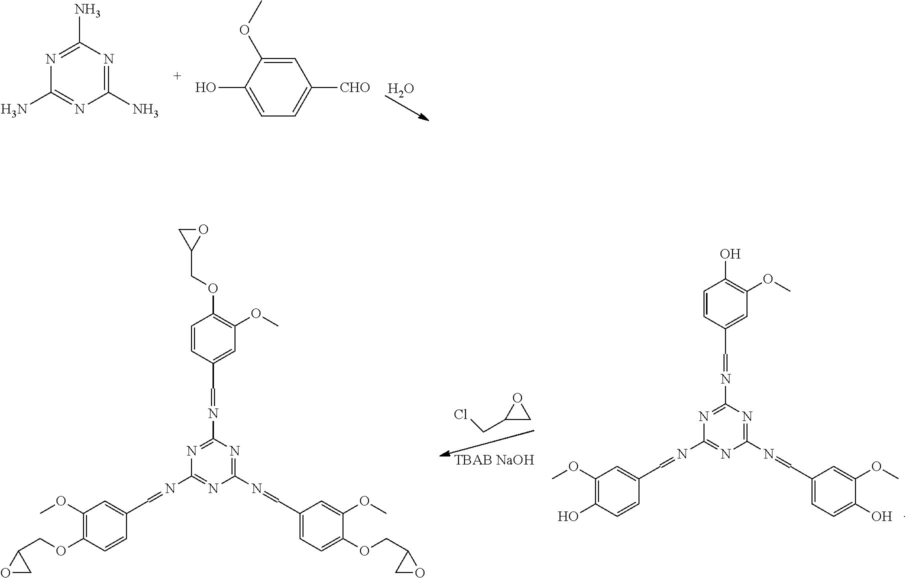

A main degradation mechanism of the degradable lost circulation material according to the present disclosure is based on the acid-base instability of a Schiff base bond in combination with properties of an epoxy resin material such as high-temperature resistance and high strength. In the present disclosure, vanillin is grafted with tripolycyanamide to produce a Schiff base bond, and the Schiff base bond could be hydrolyzed under acid/alkali conditions, which lays a foundation for the degradability of the degradable lost circulation material. The epichlorohydrin is then grafted thereon, followed by epoxidation; an epoxy group is cured with a curing agent, and the crushing is then conducted to finally obtain the degradable lost circulation material. The epoxidized vanillin Schiff base compound is a reaction intermediate. The vanillin Schiff base intermediate with the Schiff base bond is further epoxidized, and epoxy groups generated thereby are subsequently cured by a curing agent.

In some embodiments of the present disclosure, after being in a hydrochloric acid solution with a mass concentration of 3% for 24 h, a degradation rate of the degradable lost circulation material is not lower than 85% and preferably not lower than 90%; after being in a NaOH solution with a mass concentration of 3% for 36 h, a degradation rate of the degradable lost circulation material is not lower than 90% and preferably not lower than 95%.

The present disclosure also provides a method for preparing the degradable lost circulation material as described in the above technical solutions, including the following steps: [0063] mixing the epoxidized vanillin Schiff base compound and the composite curing agent, and subjecting a resulting mixture to curing reaction, to obtain a glass-like polymer; and
crushing and granulating the glass-like polymer, to obtain the degradable lost circulation material.

In the present disclosure, the epoxidized vanillin Schiff base compound and the composite curing agent are mixed, and a resulting mixture is subjected to curing reaction, to obtain a glass-like polymer.

In some embodiments of the present disclosure, the mixing is conducted in a water bath or an oil bath. In some embodiments, the water bath and the oil bath each independently have a temperature of 80° C. to 120° C. and preferably 100° C. In some embodiments, the mixing is conducted under stirring. In some embodiments, the stirring is conducted at a rate of 200 rpm to 500 rpm and preferably 300 rpm to 400 rpm. In some embodiments, the stirring is conducted for 5 min to 30 min and preferably 10 min to 20 min.

In some embodiments of the present disclosure, the curing reaction is conducted at a temperature of 100° C. to 160° C. and preferably 120° C. to 150° C. In some embodiments, the curing reaction is conducted in 2 to 4 stages and preferably in 3 stages. In some embodiments, each stage of the curing reaction is conducted for 1 h to 4 h and preferably 2 h to 3 h.

In some embodiments of the present disclosure, the curing reaction is conducted as follows: a mixture obtained by mixing the epoxidized vanillin Schiff base compound and the composite curing agent is introduced into a mold, and the mold is then placed in an oven for the curing reaction.

In some embodiments of the present disclosure, after the curing reaction is completed, a product obtained from the curing reaction is cooled to room temperature, and then demolded to obtain the glass-like polymer.

In the present disclosure, after the glass-like polymer is obtained, the glass-like polymer is crushed and granulated to obtain the degradable lost circulation material.

In the present disclosure, there is no special restriction on a process of the crushing and granulating, and a process of the crushing and granulating well known in the art may be adopted as long as the degradable lost circulation material with a desired particle size could be obtained. [0072] The present disclosure also provides use of the degradable lost circulation material as described in the above technical solutions or the degradable lost circulation material prepared by the method as described in the above technical solutions in plugging for a high-temperature fractured reservoir.

In some embodiments of the present disclosure, based on a volume of a drilling fluid, a ratio of a mass of the degradable lost circulation material to the volume of the drilling fluid is in a range of (2-15) g: 100 mL, and preferably (5-10) g: 100 mL. In some embodiments, a temperature of the high-temperature fractured reservoir is not higher than 180° C.

In the present disclosure, there is no special restriction on the use of the degradable lost circulation material in the plugging and its removal for the high-temperature fractured reservoir, and a use method well known in the art may be adopted.

The degradable lost circulation material according to the present disclosure has a low density, and exhibits excellent degradability, compressive strength, suspension stability, plugging and pressure-bearing effects, and high-temperature resistance, and great self-adaptability, and could meet the use requirements of plugging and its removal for high-temperature fractured reservoirs.

The technical solutions in the present disclosure will be described below clearly and completely in conjunction with the examples in the present disclosure, but the examples shall not be construed as limiting the scope of the present disclosure.

### Example 1

A tripolycyanamide solution (which was prepared with deionized water at 90° C. as a solvent and had a concentration of 0.3 mol/L) and a vanillin solution (which was prepared with deionized water at 90° C. as a solvent and had a concentration of 2 mol/L) were prepared separately and the two solutions were then mixed at a tripolycyanamide-to-vanillin molar ratio of 4:1. A resulting mixture was subjected to aldehyde-amine condensation reaction at 90° C. for 80 h to obtain a first reaction system. The first reaction system was filtered to obtain a solid, and the solid was washed 4 times with deionized water at 90° C. and then oven-dried at 80° C. and a vacuum degree of 0.09 MPa for 24 h to obtain a vanillin Schiff base intermediate.

The vanillin Schiff base intermediate, epichlorohydrin, and tetramethylammonium bromide were mixed at a molar ratio of 17:9:3, and a resulting mixture was subjected to a first epoxidation reaction at 88° C. for 8 h to obtain a second reaction system. A NaOH aqueous solution with a mass fraction of 23% was prepared in the same volume as the second reaction system and slowly added to the second reaction system, and a resulting mixture was subjected to a second epoxidation reaction at 70° C. for 5 h to obtain a third reaction system. The third reaction system was filtered to obtain a viscous liquid, and the viscous liquid was washed 4 times with deionized water and then subjected to rotary evaporation at 40° C. and a vacuum degree of 0.09 MPa for 2 h to obtain the epoxidized vanillin Schiff base compound.

The epoxidized vanillin Schiff base compound and a composite curing agent (which was obtained by mixing trimellitic anhydride, methylhexahydrophthalic anhydride, and 2-ethyl-4-methylimidazole at a molar ratio of 2:2:3) at a molar ratio of 40:5 were placed in a beaker, and stirred at 300 rpm at 100° C. for 20 min. A resulting mixture was then poured into a mold, and the mold was placed in a high-temperature oven, in which, the resulting mixture was subjected to crosslinking-curing separately at 110° C. 130° C., and 160° C. for 2 h, cooled to room temperature, then demolded, and crushed and granulated to obtain the degradable lost circulation material.

### Example 2

A tripolycyanamide solution (which was prepared with deionized water at 90° C. as a solvent and had a concentration of 0.3 mol/L) and a vanillin solution (which was prepared with deionized water at 90° C. as a solvent and had a concentration of 2 mol/L) were prepared separately and the two solutions were then mixed at a tripolycyanamide-to-vanillin molar ratio of 3.5:1. A resulting mixture was subjected to aldehyde-amine condensation reaction at 85° C. for 80 h to obtain a first reaction system. The first reaction system was filtered to obtain a solid, and the solid was washed 4 times with deionized water at 100° C. and then oven-dried at 80° C. and a vacuum degree of 0.09 MPa for 24 h, to obtain a vanillin Schiff base intermediate.

The vanillin Schiff base intermediate, epichlorohydrin, and tetrabutylammonium bromide were mixed at a molar ratio of 15:6:2, and a resulting mixture was subjected to a first epoxidation reaction at 90° C. for 8 h to obtain a second reaction system. A NaOH aqueous solution with a mass fraction of 20% was prepared in the same volume as the second reaction system and slowly added to the second reaction system. A resulting mixture was subjected to a second epoxidation reaction at 60° C. for 6 h to obtain a third reaction system. The third reaction system was filtered to obtain a solid, and the solid was washed 4 times with deionized water and then subjected to rotary evaporation at 40° C. and a vacuum degree of 0.09 MPa for 2 h, to obtain the epoxidized vanillin Schiff base compound.

The epoxidized vanillin Schiff base compound and a composite curing agent (which was obtained by mixing hexahydro-4-methylphthalic anhydride and 1-methylimidazole at a molar ratio of 3:2) were added at a molar ratio of 40:13 to a beaker, and stirred at 120° C. and 300 rpm for 10 min. A resulting mixture was then poured into a mold, and the mold was placed in a high-temperature oven, in which, the resulting mixture was subjected to crosslinking-curing separately at 110° C., 120° C., and 150° C. for 2 h, cooled to room temperature, then demolded, and crushed and granulated, to obtain the degradable lost circulation material.

### Example 3

A tripolycyanamide solution (which was prepared with deionized water at 90° C. as a solvent and had a concentration of 0.3 mol/L) and a vanillin solution (which was prepared with deionized water at 90° C. as a solvent and had a concentration of 2 mol/L) were prepared separately and the two solutions were then mixed at a tripolycyanamide-to-vanillin molar ratio of 2.5:1. A resulting mixture was subjected to aldehyde-amine condensation reaction at 90° C. for 24 h to obtain a first reaction system. The first reaction system was filtered to obtain a solid, and the solid was washed 4 times with deionized water at 90° C. and then oven-dried at 80° C. and a vacuum degree of 0.09 MPa for 24 h, to obtain a vanillin Schiff base intermediate.

The vanillin Schiff base intermediate, epichlorohydrin, and succinic anhydride were mixed at a molar ratio of 15:6:2, and a resulting mixture was subjected to a first epoxidation reaction at 70° C. for 9 h to obtain a second reaction system. A NaOH aqueous solution with a mass fraction of 15% was prepared in the same volume as the second reaction system and slowly added to the second reaction system. A resulting mixture was subjected to a second epoxidation reaction at 75° C. for 7 h to obtain a third reaction system. The third reaction system was filtered to obtain a solid, and the solid was washed 4 times with deionized water and then subjected to rotary evaporation at 40° C. and a vacuum degree of 0.09 MPa for 2 h, to obtain the epoxidized vanillin Schiff base compound.

The epoxidized vanillin Schiff base compound and a composite curing agent (which was obtained by mixing methyltetrahydrophthalic anhydride and 2-undecylimidazole at a molar ratio of 5:4) were added at a molar ratio of 50:28 to a beaker, and stirred at 100° C. and 300 rpm for 5 min. A resulting mixture was then poured into a mold, and the mold was placed in a high-temperature oven, in which, the resulting mixture was subjected to crosslinking-curing separately at 100° C., 130° C., and 150° C. for 1.5 h, cooled to room temperature, then demolded, and crushed and granulated, to obtain the degradable lost circulation material.

### Example 4

A tripolycyanamide solution (which was prepared with deionized water at 90° C. as a solvent and had a concentration of 0.3 mol/L) and a vanillin solution (which was prepared with deionized water at 90° C. as a solvent and had a concentration of 2 mol/L) were prepared separately and the two solutions were then mixed at a tripolycyanamide-to-vanillin molar ratio of 4.5:1. A resulting mixture was subjected to aldehyde-amine condensation reaction at 80° C. for 65 h to obtain a first reaction system. The first reaction system was filtered to obtain a solid, and the solid was washed 4 times with deionized water at 85° C. and then oven-dried at 80° C. and a vacuum degree of 0.09 MPa for 24 h to obtain a vanillin Schiff base intermediate.

The vanillin Schiff base intermediate, epichlorohydrin, and tetrabutylammonium bromide were mixed at a molar ratio of 8:5:1, and a resulting mixture was subjected a first epoxidation reaction at 90° C. for 7 h to obtain a second reaction system. A NaOH aqueous solution with a mass fraction of 22% was prepared in the same volume as the second reaction system and slowly added to the second reaction system. A resulting mixture was subjected to a second epoxidation reaction at 70° C. for 6 h to obtain a third reaction system. The third reaction system was filtered to obtain a solid, and the solid was washed 4 times with deionized water and then subjected to rotary evaporation at 40° C. and a vacuum degree of 0.09 MPa for 2 h to obtain the epoxidized vanillin Schiff base compound.

The epoxidized vanillin Schiff base compound and a composite curing agent (which was obtained by mixing hexahydro-4-methylphthalic anhydride, 1-methylimidazole, and 2-phenylimidazole at a molar ratio of 2:1:1) were added at a molar ratio of 45:22 to a beaker, and stirred at 85° C. and 300 rpm for 10 min. A resulting mixture was then poured into a mold, and the mold was placed in a high-temperature oven, in which, the resulting mixture was subjected to crosslinking-curing separately at 105° C., 125° C., and 155° C. for 2.5 h, cooled to room temperature, then demolded, and crushed and granulated, to obtain a degradable lost circulation material.

### Example 5

A tripolycyanamide solution (which was prepared with deionized water as a solvent and had a concentration of 0.3 mol/L) and a vanillin solution (which was prepared with deionized water as a solvent and had a concentration of 2 mol/L) were prepared separately and the two solutions were then mixed at a tripolycyanamide-to-vanillin molar ratio of 3:1. A resulting mixture was subjected to aldehyde-amine condensation reaction at 85° C. for 50 h to obtain a first reaction system. The first reaction system was filtered to obtain a solid, and the solid was washed 4 times with deionized water at 95° C. and then oven-dried at 80° C. and a vacuum degree of 0.09 MPa for 24 h to obtain a vanillin Schiff base intermediate.

The vanillin Schiff base intermediate, epichlorohydrin, and tetrabutylammonium bromide were mixed at a molar ratio of 17:6:3, and a resulting mixture was subjected to a first epoxidation reaction at 95° C. for 6.5 h to obtain a second reaction system. A NaOH aqueous solution with a mass fraction of 23% was prepared in the same volume as the second reaction system and slowly added to the second reaction system. A resulting mixture was subjected to a second epoxidation reaction at 68° C. for 6.5 h to obtain a third reaction system. The third reaction system was filtered to obtain a solid, and the solid was washed 4 times with deionized water and then subjected to rotary evaporation at 40° C. and a vacuum degree of 0.09 MPa for 2 h, to obtain the epoxidized vanillin Schiff base compound.

The epoxidized vanillin Schiff base compound and a composite curing agent (which was obtained by mixing trimellitic anhydride, hexahydro-4-methylphthalic anhydride, 1-methylimidazole, and 2-ethyl-4-methylimidazole at a molar ratio of 1:1:1:1) were added at a molar ratio of 42:14 to a beaker, and stirred at 105° C. and 300 rpm for 20 min. A resulting mixture was then poured into a mold, and the mold was placed in a high-temperature oven, in which, the resulting mixture was subjected to crosslinking-curing separately at 110° C., 130° C., and 155° C. for 1 h, cooled to room temperature, then demolded, and crushed and granulated to obtain the degradable lost circulation material.

### Comparative Example 1

Calcium carbonate, an acid-soluble lost circulation material most commonly used in well drilling for oil and gas reservoirs.

### Performance Testing

The degradability, compressive strength, suspension stability, and plugging performance of the degradable lost circulation materials in the examples and the acid-soluble lost circulation material in the comparative example each were evaluated.

### (1) Degradability

The degradable lost circulation materials prepared in Examples 1 to 5 and the acid-soluble lost circulation material in Comparative Example 1 each were placed in a 3% HCl solution and a 3% NaOH solution to determine the degradability of each material. Results are shown in Table 1.

TABLE 1
Experimental results of degradability evaluation

| | Degradation | Degradation | Degradation | Degradation |
|---|---|---|---|---|
| | rate after being | rate after being | rate after being | rate after being |
| | in the 3% HCl | in the 3% NaOH | in the 3% HCl | in the 3% NaOH |
| Test | solution for | solution for | solution for | solution for |
| sample | 24 h (%) | 24 h (%) | 36 h (%) | 36 h (%) |
| Example 1 | 93.1 | 53.9 | 98.5 | 98.0 |
| Example 2 | 90.5 | 47.2 | 97.9 | 97.7 |
| Example 3 | 92.4 | 51.1 | 98.8 | 98.2 |
| Example 4 | 91.2 | 48.8 | 97.9 | 97.7 |
| Example 5 | 87.5 | 43.2 | 92.5 | 90.5 |
| Comparative | 87.1 | 4.6 | 92.6 | 6.8 |
| Example 1 | | | | |

The experimental results in Table 1 show that after being in the 3% HCl solution for 24 h, degradation rates of the lost circulation materials of the present disclosure are 85% or more; after being in the 3% HCl solution for 36 h, degradation rates of the lost circulation materials are 90% or more; and after being in the 3% NaOH solution for 36 h, degradation rates of the lost circulation materials are 90% or more, indicating that these lost circulation materials are easily degraded.

### (2) Compressive Strength

A lost circulation material should have a high compressive strength and stiffness, and could reach a self-locking balance through contact occlusion and embedded filling in fractures to form a strengthened force chain network structure, thereby preventing the instability caused by squeezing and crushing and the instability caused by sliding friction. Compressive strengths of the degradable lost circulation materials prepared in Examples 1 to 5 and the acid-soluble lost circulation material (calcium carbonate) in Comparative Example 1 at a pressure of 30 MPa were evaluated experimentally. Results are shown in Table 2.

TABLE 2
Compressive crushing rates of the lost
circulation materials at 30 MPa

| | Crushing rate at room | Crushing rate after aging |
|---|---|---|
| Test sample | temperature (%) | at 180° C. (%) |
| Example 1 | 6.9 | 14.5 |
| Example 2 | 7.5 | 13.9 |
| Example 3 | 9.5 | 16.8 |
| Example 4 | 7.7 | 14.2 |
| Example 5 | 6.7 | 14.9 |
| Comparative | 48.2 | 53.8 |
| Example 1 | | |

As can be seen from Table 2, at room temperature, crushing rates of the degradable lost circulation materials in Examples 1 to 5 at a high pressure of 30 MPa are 6.7% to 9.5%, and a crushing rate of the commonly-used lost circulation material of calcium carbonate is 48.2%, indicating that the lost circulation materials of the present disclosure have a great pressure-bearing capacity meeting the application requirements under harsh high-pressure conditions, and have a much higher strength than that of the existing commonly-used acid-soluble lost circulation material of calcium carbonate. After hot aging at 180° C., under simulated deep high-temperature conditions, crushing rates of the degradable lost circulation materials in Examples 1 to 5 are 13.9% to 16.8%, which are far lower than 53.8% of calcium carbonate, indicating that the lost circulation materials of the present disclosure still have a great pressure-bearing capacity after hot aging at 180° C., and have a much higher strength than that of calcium carbonate, which could meet the application requirements under harsh deep high-temperature and high-pressure conditions.

### (3) Suspension Stability Evaluation

In order to prevent the sedimentation of a lost circulation material to adversely affect the plugging effect, the lost circulation material needs to exhibit excellent suspension stability in a drilling fluid. The suspension stability of the degradable lost circulation materials prepared in Examples 1 to 5 and the acid-soluble lost circulation material of calcium carbonate in Comparative Example 1 were tested according to the following test method: 8% of a self-degradable lost circulation material was added to a lost circulation material-carrying liquid, and the resulting mixture was thoroughly stirred, transferred to a measuring cylinder, and left to stand for 2 h such that the mixture was divided into upper and lower parts, and the lost circulation material in each part was screened out, washed, oven-dried, and weighed. A suspending rate of the lost circulation material was calculated according to Equation (1). Experimental results are shown in Table 3.

$$S = {M}_{{T}_{x}} \text{?} \qquad Equation ( 1 )$$

$$\text{?} \text{indicates text missing or illegible when filed}$$

whereSrepresents a suspending rate, %; M_{T}represents a mass of the lost circulation material in the upper part, in unit of g; and M_{B}represents a mass of the lost circulation materialin the lower part, in unit of g.

TABLE 3
Results of suspension stability evaluation of the degradable
lost circulation materials and calcium carbonate

| Test sample | Suspending rate/% |
|---|---|
| Example 1 | 91.6 |
| Example 2 | 93.4 |
| Example 3 | 93.1 |
| Example 4 | 91.3 |
| Example 5 | 92.1 |
| Comparative Example 1 | 12.8 |

The results in Table 3 show that, in the same particle size range (20 mesh to 40 mesh), compared with the calcium carbonate in Comparative Example 1, the degradable lost circulation materials with different particle sizes prepared in the present disclosure all have a suspending rate of greater than 90% due to a small density (1.14 g cm⁻³), indicating that the degradable lost circulation materials have excellent suspension stability and could effectively prevent the failure of plugging operation due to sedimentation effect of a lost circulation material.

### (4) Plugging Performance Evaluation

A long fracture plugging simulation experiment device self-developed by China University of Petroleum (East China) was used to evaluate pressure-bearing and plugging effects of 2% (18 mesh to 30 mesh)+4% (30 mesh to 60 mesh)+2% (60 mesh to 100 mesh) of lost circulation materials added in a drilling fluid for 2×1 mm wedge-shaped fractures, with clean water+0.4% polyanionic cellulose (PAC-LV)+0.2% xanthan gum (XC) as a base slurry for the drilling fluid. Results are shown in Table 4.

TABLE 4
Experimental results of fracture-plugging performance
evaluation of the lost circulation materials

| | Pressure-bearing | Filtration |
|---|---|---|
| Test sample | capacity/MPa | loss/mL |
| Example 1 | 15 | 12.5 |
| Example 2 | 14.2 | 11.0 |
| Example 3 | 14.0 | 14.8 |
| Example 4 | 13.1 | 15.3 |
| Example 5 | 14.2 | 12.9 |
| Comparative Example 1 | 13.0 | 19.5 |

Notes: Given a pressure-bearing capacity of the device, the pressurization was stopped after the pressure-bearing capacity reached 15 MPa.

As can be seen from Table 4, the degradable lost circulation materials of the present disclosure exhibit prominent plugging and pressure-bearing effects, and have a pressure-bearing capacity as high as 15 MPa, which is better than the commonly-used acid-soluble lost circulation material of calcium carbonate (Comparative Example 1).

Although the present disclosure has been described in detail through the above embodiments, the embodiments are merely some rather than all of the embodiments of the present disclosure. Other embodiments could be acquired by a person based on these embodiments without creative efforts, which shall fall within the scope of the present disclosure.

What is claimed is:

1. A degradable lost circulation material for protecting oil and gas reservoirs, comprising an epoxidized vanillin Schiff base compound and a composite curing agent,
wherein the epoxidized vanillin Schiff base compound has a structure shown in formula I: 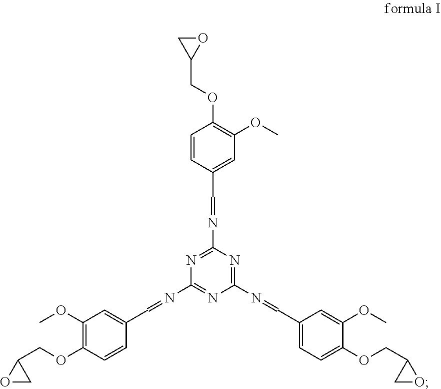 [SMILES: COc1cc(C=Nc2nc(N=Cc3ccc(OCC4CO4)c(OC)c3)nc(N=Cc3ccc(OCC4CO4)c(OC)c3)n2)ccc1OCC1CO1]
and
the composite curing agent comprises an anhydride curing agent and an imidazole curing agent.

2. The degradable lost circulation material as claimed in claim 1, wherein a molar ratio of the epoxidized vanillin Schiff base compound to the composite curing agent is in a range of (40-50):(3-28).

3. The degradable lost circulation material as claimed in claim 1, wherein the anhydride curing agent comprises at least one selected from the group consisting of phthalic anhydride, trimellitic anhydride, hexahydro-4-methylphthalic anhydride, methylhexahydrophthalic anhydride, and methyltetrahydrophthalic anhydride.

4. The degradable lost circulation material as claimed in claim 1, wherein the imidazole curing agent comprises at least one selected from the group consisting of 1-methylimidazole, 2-ethyl-4-methylimidazole, 2-undecylimidazole, and 2-phenylimidazole.

5. The degradable lost circulation material as claimed in claim 1, wherein a molar ratio of the anhydride curing agent to the imidazole curing agent is in a range of (1-5):(1-4).

6. The degradable lost circulation material as claimed in claim 2, wherein a molar ratio of the anhydride curing agent to the imidazole curing agent is in a range of (1-5):(1-4).

7. The degradable lost circulation material as claimed in claim 3, wherein a molar ratio of the anhydride curing agent to the imidazole curing agent is in a range of (1-5):(1-4).

8. The degradable lost circulation material as claimed in claim 4, wherein a molar ratio of the anhydride curing agent to the imidazole curing agent is in a range of (1-5):(1-4).

9. The degradable lost circulation material as claimed in claim 1, wherein the epoxidized vanillin Schiff base compound is prepared by a process comprising the steps of
subjecting vanillin and tripolycyanamide to aldehyde-amine condensation reaction in a solution, to obtain a vanillin Schiff base intermediate, wherein the vanillin Schiff base intermediate has a structure shown in formula II: 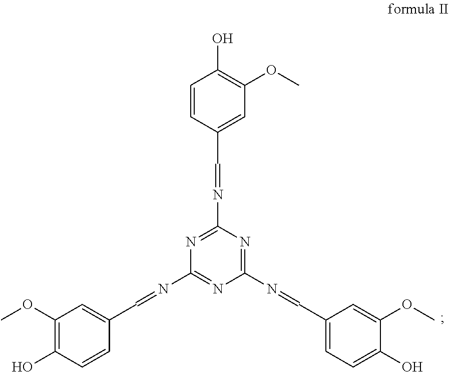
mixing the vanillin Schiff base intermediate, epichlorohydrin, and a first catalyst, and subjecting a resulting mixture to a first epoxidation reaction, to obtain a first epoxidation product; and
mixing the first epoxidation product with a second catalyst, and subjecting a resulting mixture to a second epoxidation reaction, to obtain the epoxidized vanillin Schiff base compound.

10. The degradable lost circulation material as claimed in claim 9, wherein the aldehyde-amine condensation reaction is conducted at a temperature of 70° C. to 90° C. for 24 h to 96 h.

11. The degradable lost circulation material as claimed in claim 9, wherein the first epoxidation reaction is conducted at a temperature of 70° C. to 95° C. for 6 h to 9 h.

12. A method for preparing the degradable lost circulation material as claimed in claim 1, comprising the steps of
mixing the epoxidized vanillin Schiff base compound and the composite curing agent, and subjecting a resulting mixture to curing reaction, to obtain a glass-like polymer; and
crushing and granulating the glass-like polymer, to obtain the degradable lost circulation material.

13. A method for plugging in a high-temperature fractured reservoir, comprising
adding the degradable lost circulation material as claimed in claim 1 to a drilling fluid used.

14. The method as claimed in claim 13, wherein a temperature of the high-temperature fractured reservoir is not higher than 180° C.

15. The method as claimed in claim 13, wherein a ratio of a mass of the degradable lost circulation material to a volume of the drilling fluid is in a range of (2-15) g: 100 mL.
